(19) United States
(12) Patent Application Publication
(10) Pub. No.: US 2017/0156834 A1
(43) Pub. Date: Jun. 8, 2017

(54) Ultrasound Scaler Tip and Ultrasound Scaler

(71) Applicant: Minoru KANNO, Sendai-shi, Miyagi (JP)

(72) Inventors: Minoru KANNO, Sendai-shi (JP); Taro KANNO, Sendai-shi (JP)

(73) Assignee: Minoru KANNO, Sendai-shi, Miyagi (JP)

(21) Appl. No.: 15/309,704

(22) PCT Filed: May 8, 2014
(86) PCT No.: PCT/JP2014/062314

§ 371 Date: Nov. 8, 2016

(51) Int. Cl. A61C 17/16; A61L 2/18; A61C 1/08; A61C 17/20; A61C 17/02

(52) U.S. Cl. CPC A61C 17/16; A61C 17/20; A61C 17/02; A61C 1/088; A61L 2/18

(57) ABSTRACT

Ultrasound scaler tip and ultrasound scaler has high sterilization effect. Toothbrush has reduced effect on mouth interior. An ultrasound scaler tip has tip body and optical fiber. Tip body has tubular shape with pointed-end opening and rear-end opening, rear end that can be attached to ultrasound scaler body, and intermediate curved part. Optical fiber is thinner than minimum inner diameter of tip body and has rear end toward which light from light source can enter. Optical fiber is inserted from rear-end opening to pointed-end opening of tip body, and has intermediate part that is in contact with curved part, so that end is vibrated by ultrasonic vibration of tip body. Ultrasound scaler body houses liquid supply device, ultrasound-generating device, and light source, which is attached to rear end of ultrasound scaler tip. Light source causes light for sterilization in predetermined wavelength range to enter rear end of optical fiber.

### TECHNICAL FIELD

The present invention relates to an ultrasound scaler tip and an ultrasound scaler.

### BACKGROUND ART

Conventionally, a ultrasound scaler is known (for example, see Japanese Unexamined Patent Application Publication No. 2012-235979 A), which crushes calculus by transmitting ultrasonic vibration generated by an ultrasonic transducer to the pointed-end part of a probe, supplies an antiseptic solution supplied from a liquid supply means from the pointed-end part of the probe to tooth parts, and then irradiates the supplied antiseptic solution with light directed by a light guide from a light source apparatus, so as to generate hydroxyl radicals for sterilization.

### CITATION LIST

### Patent Literature

Japanese Unexamined Patent Application Publication No. 2012-235979 A

### SUMMARY OF INVENTION

### Technical Problem

In the case of the ultrasound scaler disclosed in JP Patent Publication (Kokai) No. 2012-235979 A, hydroxyl radicals are generated to perform sterilization, so that a dental scaling part is irradiated with light of the light guide. However, this ultrasound scaler is problematic in that the probe has a thin opening at the pointed end, the area to be irradiated with an even thinner light beam of the light guide is narrow, the area where hydroxyl radicals are generated is limited, and thus the sterilization effect cannot be exhibited sufficiently.

The present invention has been achieved noting such problems and an objective of the present invention is to provide an ultrasound scaler tip and an ultrasound scaler having a high sterilization effect.

### Solution to Problem

To achieve the above objectives, the ultrasound scaler tip according to the present invention is an ultrasound scaler tip, which is used while being attached to an ultrasound scaler body having a light source of light for sterilization in a predetermined wavelength range, and has a tip body and an optical fiber, wherein: the tip body has a tubular shape with a pointed-end opening and a rear-end opening, a rear end that can be attached to the ultrasound scaler body, and an intermediate curved part; and the optical fiber is thinner than the minimum inner diameter of the tip body, has a rear end toward which light can enter from the light source, is inserted from the rear-end opening to the pointed-end opening of the tip body, and has an intermediate part that is in contact with the curved part, so that the pointed end is vibrated by ultrasonic vibration of the tip body, and has flexibility and projects from the pointed-end opening, so that the vibration width of the end of the optical fiber is increased.

The ultrasound scaler tip according to the present invention is used with the rear end attached to the ultrasound scaler body. The tip body crushes and removes calculus within the periodontal pocket by ultrasonic vibration. The optical fiber is used for irradiation with light for sterilization within the range where the pointed end is vibrated by the ultrasonic vibration of the tip body. Therefore, the optical fiber enables a wider range of light irradiation compared with a case where the optical fiber is fixed to the tip body, so as to be able to enhance the sterilization effect. The optical fiber has the intermediate part that is in contact with the curved part of the tip body, which ensures the transmission of the ultrasonic vibration of the tip body to the optical fiber.

Light for sterilization in a predetermined wavelength range is preferably light having a wavelength between 400 nm and 420 nm. Light irradiation is preferably performed with the optical fiber in the presence of a hydrogen peroxide solution or hypochlorous acid in order to enhance the sterilization effect. The curved part of the tip body may also have a curved projection located to be in contact with the optical fiber. In this case, the optical fiber comes into contact with or comes out of the projection to increase its vibration width, so that the range of light irradiation, that is, the sterilization range can be more increased.

The tip body of the ultrasound scaler tip according to the present invention preferably has a plane symmetrical shape, a pointed-end part that can be inserted into a periodontal pocket, and a pair of projecting parts that extend to the outer face of the pointed-end part along the longitudinal direction and project vertically with respect to the symmetrical surfaces of the tip body. In this case, calculus can be effectively crushed and removed with the projecting parts.

The pointed end of the optical fiber projects from the pointed-end opening. Therefore, the vibration width of the pointed end of the optical fiber is increased, so that the range of light irradiation, that is, the sterilization range can be more increased.

The ultrasound scaler according to the present invention is an ultrasound scaler having the ultrasound scaler tip, which has an ultrasound scaler body, a liquid supply means, an ultrasound-generating means, and a light source, wherein: the ultrasound scaler body houses the liquid supply means, the ultrasound-generating means and the light source, and is attached to the rear-end opening of the ultrasound scaler tip, the liquid supply means is provided so as to be able to supply liquid to the rear-end opening of the tip body and to release the liquid from the pointed-end opening; the ultrasound-generating means is provided to generate ultrasound so as to ultrasonically vibrate the tip body; and the light source is provided so that light for sterilization in a predetermined wavelength range enters the rear end of the optical fiber.

The ultrasound scaler according to the present invention ultrasonically vibrates the tip body by the ultrasound-generating means, and then crushes and removes calculus within the periodontal pocket with the ultrasonic vibration of the tip body. The end of the optical fiber is vibrated by the ultrasonic vibration of the tip body, and then irradiation with light for sterilization is performed from the light source within the vibration range. Therefore, the optical fiber enables a wider range of light irradiation and sterilization compared with a case where the optical fiber is fixed to the tip body. The optical fiber has the intermediate part that comes in contact with the curved part of the tip body, which ensures the transmission of the ultrasonic vibration of the tip body to the optical fiber. Liquid is released by the liquid supply means from the pointed-end opening of the tip body. An antiseptic solution is used as such a liquid, and thus disinfection can be performed together with dental scaling. In particular, light having a wavelength between 400 nm and 420 nm is used as light of the light source, and a hydrogen peroxide solution or hypochlorous acid having a concentration of 3 mass % or less is used as liquid to be supplied from the liquid supply means, so that the sterilization effect can be enhanced safely.

The ultrasound scaler according to the present invention has the ultrasound scaler tip having projecting parts on the outer face of the pointed-end part of the tip body, wherein the ultrasound-generating means preferably has a configuration by which the tip body is ultrasonically vibrated vertically to the longitudinal direction of and vertically to the projecting direction of the projecting parts. In this case, calculus can be effectively crushed and removed using the projecting parts in accordance with the direction of the ultrasonic vibration of the tip body.

The toothbrush according to the present invention has a handle, a head, and a brush, wherein: the head has LED for irradiation with light having a wavelength between 400 nm and 420 nm within the head and provided at an end of the handle; and the brush is made of an optical fiber, and is provided on the head so that the LED light enters and the light that has entered goes out from the end.

The toothbrush according to the present invention is capable of performing irradiation with light having a wavelength between 400 nm and 420 nm from the end of the brush made of an optical fiber when a user holds the handle for toothbrushing. Keeping a hydrogen peroxide solution or hypochlorous acid having a concentration of 3 mass % or less in the mouth followed by toothbrushing makes it possible to generate hydroxyl radicals at locations irradiated with light and to sterilize the locations. Light having a wavelength between 400 nm and 420 nm has a reduced effect on the interior of the mouth and can sterilize the same more safely, compared with ultraviolet rays.

### Advantageous Effects of Invention

According to the present invention, the ultrasound scaler tip and the ultrasound scaler having a high sterilization effect can be provided.

### BRIEF DESCRIPTION OF DRAWINGS

FIG. 1 is a side view showing an ultrasound scaler tip according to an embodiment of the present invention.

FIG. 2 is an enlarged plan view showing the pointed-end part of the ultrasound scaler tip shown in FIG. 1.

FIG. 3 is an illustration showing the structure of an ultrasound scaler according to an embodiment of the present invention.

### DESCRIPTION OF EMBODIMENTS

Embodiments of the present invention are described below on the basis of drawings.

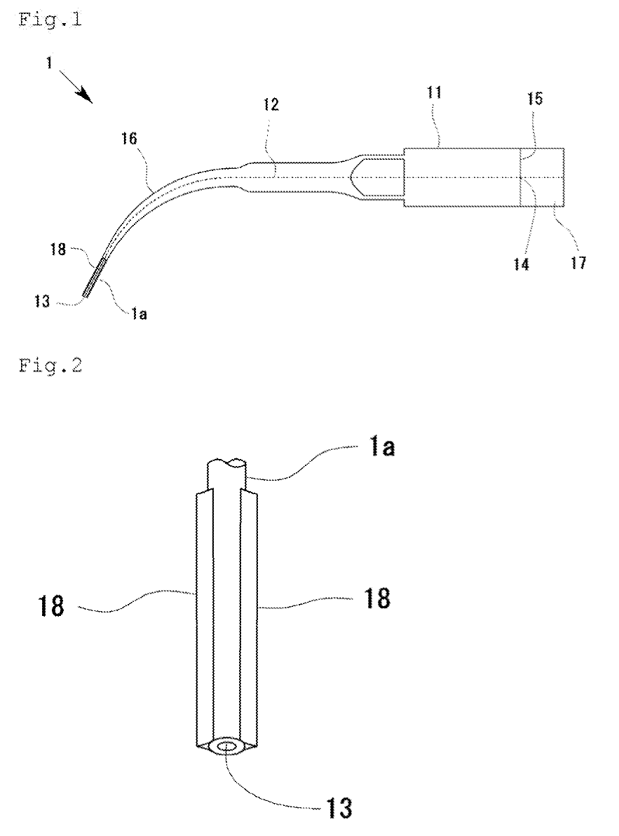

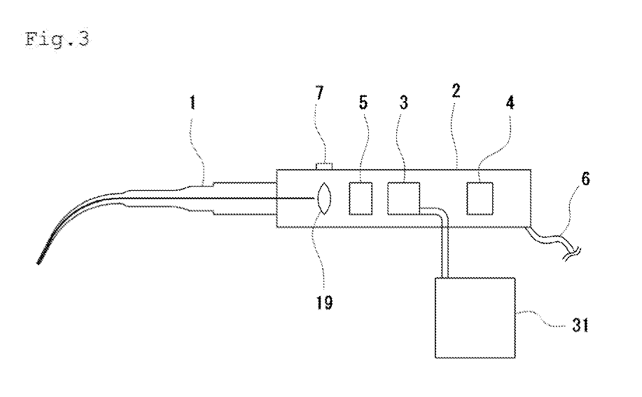

FIG. 1 shows an ultrasound scaler tip according to an embodiment of the present invention and FIG. 2 shows the pointed-end part thereof. FIG. 3 shows an ultrasound scaler having the ultrasound scaler tip.

As shown in FIG. 3, the ultrasound scaler has an ultrasound scaler tip 1, an ultrasound scaler body 2, a liquid supply means 3, an ultrasound-generating means 4, a light source 5, a power supply 6, and a switch 7.

As shown in FIG. 1, the ultrasound scaler tip 1 has a tip body 11 and an optical fiber 12. The tip body 11 has a tubular shape with a pointed-end opening 13 and a rear-end opening 14. The tip body 11 has a rear end 15 that can be attached to the ultrasound scaler body 2 and an intermediate curved part 16. The tip body 11 has the rear end 15 to which a fiber adapter 17 is attached. The tip body 11 has a plane symmetrical shape, and a thin pointed-end part 1a that can be inserted into a periodontal pocket. As shown in FIG. 2, the tip body 11 extends to the outer face of the pointed-end part 1a along the longitudinal direction, and has a pair of fin-shaped projecting parts 18 projecting vertically with respect to the symmetrical surfaces of the tip body 11.

The optical fiber 12 is thinner than the minimum inner diameter of the tip body 11, and has the rear end 15 toward which light can enter from the light source 5. The optical fiber 12 has flexibility, and is inserted from a fiber adapter 17 attached to the rear-end opening 14 to the pointed-end opening 13 of the tip body 11. The optical fiber 12 is disposed leaving a space around and between the fiber and the tip body 11 at the pointed-end opening 13 of the tip body 11. The optical fiber 12 has the intermediate part that is in contact with the curved part 16, so that the end is vibrated as a result of the ultrasonic vibration of the tip body 11.

The ultrasound scaler body 2 houses the liquid supply means 3, the ultrasound-generating means 4 and the light source 5. The ultrasound scaler body 2 has an end attached to the rear end 15 of the ultrasound scaler tip 1. The liquid supply means 3 is provided so as to be able to supply liquid contained within a liquid tank 31 located outside the ultrasound scaler body 2 to the rear-end opening 14 of the tip body 11 and to release the liquid from the pointed-end opening 13. As the liquid, a hydrogen peroxide solution or hypochlorous acid having a concentration of 3 mass % or less is used appropriately.

The ultrasound-generating means 4 is provided to generate ultrasound, so as to ultrasonically vibrate the tip body 11. The ultrasound-generating means 4 has a configuration by which the tip body 11 is ultrasonically vibrated vertically to the longitudinal direction of and vertically (the arrow direction in FIG. 1) to the projecting direction of the projecting parts 18.

The light source 5 is made of LED, and is provided so that light for sterilization in a predetermined wavelength range enters in the form of a laser beam the rear end 15 of the optical fiber 12 via a lens 19. Light for irradiation from the light source 5 is light having a wavelength between 400 nm and 420 nm.

The power supply 6 is composed of a 100V power supply, and is connected to supply electric power to the liquid supply means 3, the ultrasound-generating means 4, and the light source 5 for actuation or light emission. The power supply 6 can be turned on and off via the operation of the switch 7.

Next, the action is explained.

The ultrasound scaler according to the present invention is used for dental treatment, and is specifically used with the pointed-end part la of the tip body 11 inserted in a periodontal pocket. When the switch 7 is turned on, the tip body 11 is ultrasonically vibrated by the ultrasound-generating means 4, so as to crush and remove calculus within the periodontal pocket via the ultrasonic vibration of the tip body 11. The ultrasound-generating means 4 ultrasonically vibrates the tip body 11 vertically to the longitudinal direction of and vertically to the projecting direction of the projecting parts 18. In this manner, calculus can be effectively crushed and removed using the projecting parts 18 in accordance with the direction of the ultrasonic vibration of the tip body 11.

The end of the optical fiber 12 is vibrated by the ultrasonic vibration of the tip body 11, irradiation with light for sterilization is performed from the light source 5 within the vibration range. Therefore, the optical fiber 12 enables a wider range of light irradiation compared with a case where the optical fiber is fixed to the tip body 11, so as to be able to enhance the sterilization effect. The optical fiber 12 has the intermediate part that is in contact with the curved part 16 of the tip body 11, which ensures the transmission of the ultrasonic vibration of the tip body 11 to the optical fiber 12. Liquid is released into the periodontal pocket from the pointed-end opening 13 of the tip body 11 by the liquid supply means 3, so that disinfection can be performed together with dental scaling. The light having a wavelength between 400 nm and 420 nm of the light source 5 reacts with the hydrogen peroxide solution or hypochlorous acid having a concentration of 3 mass % or less to generate hydroxyl radicals. In this manner, sterilization can be performed safely for human bodies and the growth of periodontal disease bacteria can be suppressed.

### REFERENCE SIGNS LIST

1 Ultrasound scaler tip
1a Pointed-end part
2 Ultrasound scaler body
3 Liquid supply means
4 Ultrasound-generating means
5 Light source
6 Power supply
7 Switch
11 Tip body
12 Optical fiber
13 Pointed-end opening
14 Rear-end opening
16 Curved part
18 Projecting part

1. An ultrasound scaler tip, which is used while being attached to an ultrasound scaler body having a light source of light for sterilization in a predetermined wavelength range, and has a tip body and an optical fiber, wherein:
said tip body has a tubular shape with a pointed-end opening, a rear-end opening, a rear end that can be attached to the ultrasound scaler body, and an intermediate curved part; and
said optical fiber is thinner than the minimum inner diameter of said tip body, has a rear end toward which light can enter from said light source, is inserted from said rear-end opening to said pointed-end opening of said tip body, has an intermediate part that is in contact with said curved part so that the pointed-end is vibrated by the ultrasonic vibration of said tip body, and has flexibility and projects from said pointed-end opening, so that the vibration width of the end of said optical fiber is increased.

2. The ultrasound scaler tip according to claim 1, wherein said tip body has a plane symmetrical shape, has a pointed-end part that can be inserted into a periodontal pocket, extends to the outer face of said pointed-end part along the longitudinal direction, and has a pair of projecting parts projecting vertically with respect to symmetrical surfaces of the tip body.

3. (canceled)

4. An ultrasound scaler, which has the ultrasound scaler tip according to claim 1, and has
an ultrasound scaler body, a liquid supply means, an ultrasound-generating means, and a light source, wherein:
said ultrasound scaler body houses said liquid supply means, said ultrasound-generating means, and said light source, and is attached to said rear end of said ultrasound scaler tip;
said liquid supply means is provided so that liquid can be supplied to said rear-end opening of said tip body and then released from said pointed-end opening;
said ultrasound-generating means is provided so as to generate ultrasound and ultrasonically vibrate said tip body; and
said light source is provided so that light for sterilization in a predetermined wavelength range enters the rear end of said optical fiber.

5. The ultrasound scaler according to claim 4, which has the ultrasound scaler tip wherein said tip body has a plane symmetrical shape, has a pointed-end part that can be inserted into a periodontal pocket, extends to the outer face of said pointed-end part along the longitudinal direction, and has a pair of projecting parts projecting vertically with respect to symmetrical surfaces of the tip body.
wherein said ultrasound-generating means has a configuration, by which said tip body is ultrasonically vibrated vertically to the longitudinal direction of and vertically to the projecting direction of said projecting parts.

6. (canceled)

7. An ultrasound scaler, which has the ultrasound scaler tip according to claim 2, and has
an ultrasound scaler body, a liquid supply means, an ultrasound-generating means, and a light source, wherein:
said ultrasound scaler body houses said liquid supply means, said ultrasound-generating means, and said light source, and is attached to said rear end of said ultrasound scaler tip;
said liquid supply means is provided so that liquid can be supplied to said rear-end opening of said tip body and then released from said pointed-end opening;
said ultrasound-generating means is provided so as to generate ultrasound and ultrasonically vibrate said tip body; and
said light source is provided so that light for sterilization in a predetermined wavelength range enters the rear end of said optical fiber.
